(19) United States
(12) Patent Application Publication
(10) Pub. No.: US 2022/0088603 A1
(43) Pub. Date: Mar. 24, 2022

(54) Bioreactor

(71) Applicant: National Central University, Taoyuan City (TW)

(72) Inventors: CHIA-WEN TSAO, Taoyuan City (TW); WEI-WEN HU, Taoyuan City (TW)

(73) Assignee: National Central University, Taoyuan City (TW)

(21) Appl. No.: 17/078,151

(22) Filed: Oct. 23, 2020

(30) Foreign Application Priority Data
Sep. 24, 2020 (TW) 109133086

(51) Int. Cl. B01L 3/00; G01N 33/533

(52) U.S. Cl. CPC B01L 3/502769; B01L 2300/0819; B01L 2400/0487; G01N 33/533

(57) ABSTRACT

The present invention relates to a bioreactor. The bioreactor includes a fluidic channel layer including a set of channels configured to generate a suction caused by a negative pressure or a retrieval force caused by a positive pressure; an elastic conductive layer configured with a pair of electrodes, configured on the fluidic channel layer, driven by the suction or the retrieval force to have a deformation toward a deformation direction, and receiving a voltage difference by the pair of the electrodes to form an electrical field along an electrical field direction; and a culture layer configured on the elastic conductive layer and providing for a biological tissue to culture in vitro on the elastic conductive layer.

### CROSS-REFERENCE TO RELATED APPLICATION

This application claims priority benefit to Taiwan Invention Patent Application Serial No. 109133086, filed on Sep. 24, 2020, in Taiwan Intellectual Property Office, the entire disclosures of which are incorporated by reference herein.

### FIELD

The present invention relates to a bioreactor that is capable of providing an electrical stimulation and a mechanical stimulation separately, alternatively or simultaneously for cells cultured in vitro, and selectively altering a degree of surface hydrophilicity in response to a change of a temperature.

### BACKGROUND

The tissue engineering is a technology using bioactive substances, and in vitro culturing or constructing methods, to culture cells and tissues in vitro for research purpose and for rebuilding or repairing organs and tissues.

In organisms, the electrical stimulation on muscle cell such as skeletal muscle by nerve cells is an important factor leading to the development and maturation of skeletal muscle. However, for in vitro culture, mechanical stress stimulation is the most important factor in the growth, maturation, differentiation, protein expression, and functionalization of muscle cells; furthermore, it has also been proven that the mechanical stimulation is able to induce the orderly arrangement of cells, promote the parallel arrangement of myotube differentiation, and enhance myotube maturity.

In conventional technology, the devices capable of stimulating cells cultured in vitro are roughly divided into two categories with different structure and design principles. The first type of the device uses microchannels to form a bionic model with hydrostatic pressure and shearing stress, and the other type of device is a machine using a mechanical stage to directly stretch a biological membrane or a mold.

However, the bionic model based on micro-channels is suitable for few biological samples only and unable to perform biological analysis; the mechanical stage provides sufficient cell culture space, but the mechanical stage has too high price, large size and great weight, and it is not easy to place the mechanical stage in a cell culture box for aseptic processing and tissue culture; that is, the mechanical stage has poor performance in space utilization. As a result, both types of conventional devices have obvious drawbacks.

The other type of device is the microchannel-based microminiaturized bioreactor using micro-system manufacturing process. The bioreactor has a reduced size and is easily placed into the cell culture box, but the cultivation space of the bioreactor is limited and causes difficulty in collecting biological samples.

There is a bioreactor with a larger size than the miniaturized bioreactor, and the bioreactor deforms the biological membrane, where the cells is cultured, with air pressure by the flow channel layer of moderate volume. However, the culture area of this bioreactor is limited to be circular, and when the circular membrane is bulged or compressed by force, the distribution of the arched deformation on the membrane is generally non-uniform, and it causes inconsistent stimulation to the cells and further affects the experimental results.

The Hosseini team used hydraulic pressure to expand the PDMS film to apply mechanical stimulation to the cells, and then doped symmetrically-shaped nano-silver into the PDMS film as conductor, so that the cells are applied with electric field stimulation when being energized. Although the above-mentioned device can apply both of electrical and mechanical stimulations on the cells, the device can perform morphological analysis for the cells on the electrodes only, and the arched deformation caused by expansion cannot stretch the cells uniformly.

Most of the above-mentioned conventional devices are designed to apply mechanical stimulation to tissues cultured in vitro, and only a few conventional devices can perform the combination of the mechanical stimulation and the electrical stimulation to tissues. Therefore, the development of the device providing combined stimulations is still in initial stage and has many problems to be overcome.

On the other hand, few studies have pointed out that the combination of electrical stimulation and mechanical stimulation can achieve the multiplier effect on the various responses produced by the tissue. Furthermore, applying combined physical stimulations to the tissue cultured in vitro is critical for maturation of tissues and is also a very important research topic.

In view of the criticality of combined stimulations, and the few amount and drawbacks of the devices capable of combining stretching stimulation and electric stimulation, which is performed by energizing cells through the substrate, in the field of tissue engineering, the inventors develop a bioreactor providing combined stimulations according to multiple tests and research, to solve the above-mentioned issues.

Hence, there is a need to solve the above deficiencies/issues.

### SUMMARY

In order to well solve the above-mentioned conventional issues, the present invention provides a bioreactor capable of performing at least one of electrical stimulation and mechanical stimulation to the biological tissue, which is cultured and attached to the culture membrane, at separate times or at the same time, and capable of altering hydrophilicity of the culture membrane in response to a change of a temperature. The entire bioreactor is developed and designed on the basis of full biocompatibility, and has simple design and benefit in space and cost; furthermore, the bioreactor has a culture zone for providing a large culture space and is able to stretch the culture membrane quite uniformly and apply the uniform combined stimulations to biological tissue. The bioreactor in accordance with the present invention has advantages of low cost and convenience in production, automation, easy to operate, and high integration.

The bioreactor in accordance with the present invention includes multiple culture zones which have large-area and are separate and independent from each other in structure, so that an operator can perform multiple biological experiments with reduced experimental errors and sufficient biological samples under exactly the same conditions. Furthermore, the bioreactor in accordance with the present invention also provides the uniform and stable stretching or electrical stimulation, and can alert the parameters of the electrical stimulation or stretching stimulation to implement dual stretching/electrical stimulations upon requirement. Furthermore, if necessary, the operator can selectively and easily obtain cell sheet in the late stage of differentiation by a cooling manner.

Accordingly, the present invention provides a bioreactor. The bioreactor includes a fluidic channel layer including a set of channels configured to generate a suction caused by a negative pressure or a retrieval force caused by a positive pressure; an elastic conductive layer configured with a pair of electrodes, configured on the fluidic channel layer, driven by the suction or the retrieval force to have a deformation toward a deformation direction, and receiving a voltage difference by the pair of the electrodes to form an electrical field along an electrical field direction; and a culture layer configured on the elastic conductive layer and providing for a biological tissue to culture in vitro on the elastic conductive layer.

The present invention further provides a bioreactor. The bioreactor includes a fluidic channel layer including a set of channels configured to generate a suction caused by a negative pressure or a retrieval force caused by a positive pressure; an elastic conductive layer configured with a pair of electrodes, configured on the fluidic channel layer, driven by the suction or the retrieval force to have a deformation toward a deformation direction, receiving a voltage difference by the pair of the electrodes to form an electrical field along an electrical field direction, and altering a degree of surface hydrophilicity in response to a change of a temperature; and a culture layer configured on the elastic conductive layer and providing for a biological tissue to culture in vitro on the elastic conductive layer.

The above content described in the summary is intended to provide a simplified summary for the presently disclosed invention, so that readers are able to have an initial and basic understanding to the presently disclosed invention. The above content is not aimed to reveal or disclose a comprehensive and detailed description for the present invention, and is never intended to indicate essential elements in various embodiments in accordance with the present invention, or define the scope or coverage in accordance with the present invention.

### DESCRIPTION OF THE DRAWINGS

A more complete appreciation of the invention and many of the attendant advantages thereof are readily obtained as the same become better understood by reference to the following detailed description when considered in connection with the accompanying drawing, wherein:

FIGS. 1 to 9 are schematic diagrams illustrating an assembly process, according to a first embodiment of a bioreactor in accordance with the present invention;

FIG. 10 is an exploded diagram illustrating most structural parts of a biocompatible reaction module, according to a second embodiment in accordance with the present invention;

FIG. 11 is a schematic diagram illustrating a structural assembling operation for the biocompatible reaction module, according to the second embodiment in accordance with the present invention;

FIG. 12 is an exploded diagram illustrating most structural parts of a biocompatible reaction module, according to a third embodiment in accordance with the present invention;

FIG. 13 is a schematic diagram illustrating a structural assembling operation of the biocompatible reaction module, according to the third embodiment in accordance with the present invention;

FIG. 14 is a schematic diagram illustrating the connection and communication between the bioreactor in accordance with the present invention and an external fluid driving equipment;

FIGS. 15 to 17 are a series of schematic cross-sectional diagrams acquired along a section line SS' shown in FIG. 13 showing an operating process of a biocompatible reaction module of a bioreactor in accordance with the present invention;

FIG. 18 is a microscopic image observed by using an inverted microscope with 4× objective lens and showing the biological tissue cell cultured on the elastic conductive layer of the bioreactor in accordance with the present invention;

FIG. 19 is a microscopic image observed by using an inverted microscope with 10× objective lens and showing the biological tissue cell cultured on the elastic conductive layer of the bioreactor in accordance with the present invention;

FIG. 20 is a schematic diagram illustrating a biocompatible reaction module generating the combined stimulation in an electrical field direction substantively parallel to the deformation direction;

FIG. 21 is a schematic diagram illustrating the biocompatible reaction module generating the combined stimulation in an electrical field direction perpendicular to the deformation direction, according to the present invention;

FIG. 22 is a microscope image showing arrangement of array of fluorescent markers before the elastic conductive layer printed with the array of fluorescent markers is stretched; and

FIG. 23 is a microscope image showing the change in arrangement of the array of fluorescent markers after the elastic conductive layer printed with the array of fluorescent markers is stretched.

### DETAILED DESCRIPTION

The present disclosure will be described with respect to particular embodiments and with reference to certain drawings, but the disclosure is not limited thereto but is only limited by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice.

It is to be noticed that the term “including”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device including means A and B” should not be limited to devices consisting only of components A and B.

The disclosure will now be described by a detailed description of several embodiments. It is clear that other embodiments can be configured according to the knowledge of persons skilled in the art without departing from the true technical teaching of the present disclosure, the claimed disclosure being limited only by the terms of the appended claims.

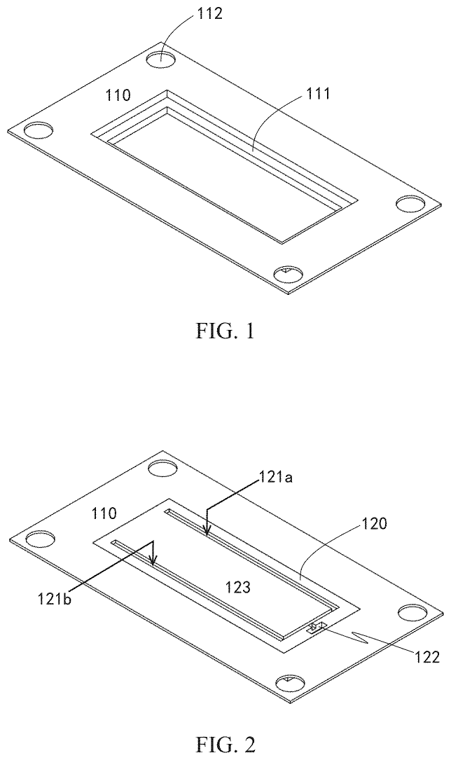

FIGS. 1 to 9 are schematic diagrams illustrating an assembly process, according to a first embodiment of a bioreactor in accordance with the present invention. In the first embodiment, the bioreactor in accordance with the present invention includes a plurality of pre-made basic components, and these basic components are configured with various structures including central slots, assembling holes or culture wells. According to the operator's operating experience and assembly proficiency, the operator generally takes about 5 to 15 minutes to assemble these basic components into the bioreactor 100 in accordance with the present invention.

Initially, a lower base 110 is provided as shown in FIG. 1. The lower base 110 preferably includes a central slot 111 for the fluidic channel layer to embedded therein, and a plurality of assembling holes 112. A part of the central slot 111 corresponds in structure to the fluidic channel layer. The fluidic channel layer 120 is embedded into the central slot 111 to mount on the lower base 110, as shown in FIG. 2, and the fluidic channel layer 120 includes microchannels 121a and 121b, a chamber 122 and an experiment and observation platform 123 connected with each other, and the microchannels 121a and 121b are connected to the external fluid driving equipment through the chamber 122; for example, the external fluid driving equipment can be an air pump, a liquid pump, a syringe, a hydraulic controller, an injection pump or a peristaltic pump, etc.

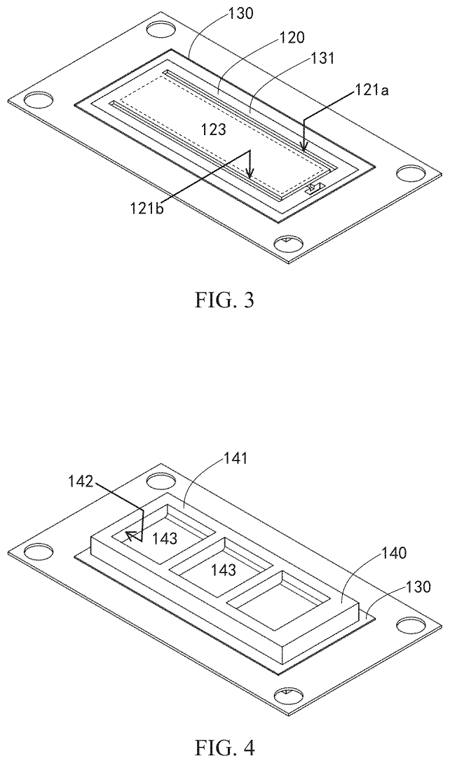

As shown in FIG. 3, then the fluidic channel layer 120 is bonded with an elastic conductive layer 130, which is light-transmittable. Preferably, the elastic conductive layer 130 and the fluidic channel layer 120 are bonded by plasma bonding, and the bonding range includes the outer sides of the microchannels 121a and 121b and a peripheral area 131, which is located from the outer sides to the edge, but the bonding range does not include the part of the elastic conductive layer 130 located on the experiment and observation platform 123. The part of the elastic conductive layer 130 located on the experiment and observation platform 123 must remain slidable with respect to the fluidic channel layer 120, so as to generate the stretch amount (or called strain). A little silicone oil can be applied as lubricant coated between the experiment and observation platform 123 and the elastic conductive layer 130, and the deformation direction of stretch is perpendicular to the microchannel 121a or 121b.

The elastic conductive layer 130 includes a sheet of elastic bio-substrate as a substrate, and an organic conductive membrane formed by depositing conductive polymeric material on the surface layer of the elastic bio-substrate by implementing a polymer polymerization process. For example, the elastic bio-substrate can be preferably made by polydimethylsiloxane (PDMS) membrane; preferably, the conductive polymeric material is selected from one of polypyrrole (PPy), polythiophene (PT), polyaniline (PANi), polyphenylene sulfide (PPS) and a combination thereof. The above-mentioned conductive polymeric materials have biocompatibility, stable properties, and conductive properties, and is suitable for cell to attach, migrate, proliferate and differentiate when being used as membrane substrate for culture in vitro. The elastic conductive layer 130 is basically a composite polymer membrane which is conductive, extensible and light-transmittable,

As shown in FIG. 4, a culture layer 140 is adhered on the elastic conductive layer 130; preferably, the elastic conductive layer 130 is adhered to the culture layer 140 by using biocompatible material such as PDMS as adhesive. In structure, the culture layer 140 includes a plurality of frame structures 141, and a plurality of well-shaped structures formed of multiple vertical walls 142 on the plurality of frame structures 141. After the culture layer 140 and the elastic conductive layer 130 are adhered to each other, the vertical walls 142 and the elastic conductive layer 130 together form a plurality of culture wells 143, which is also called culture zones. The elastic conductive layer 130 located in each culture well 143 can be used to culture cell or biological tissue in vitro thereon. The culture wells 143 are isolated from each other by the plurality of frame structures 141, to form culture zones independent from each other.

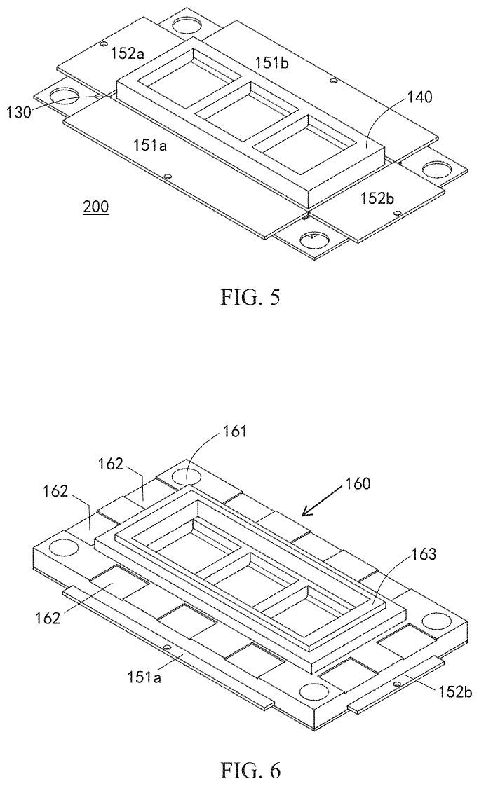

FIG. 5 depicts two pairs of electrodes connected to the surrounding edges of the elastic conductive layer 130. The two pair of electrodes include the first pair of electrodes 151a and 151b, and the second pair of electrodes 152a and 152b. The first pair of electrodes 151a and 151b and the second pair of electrodes 152a and 152b are electrically connected to an external electronic controller. The electronic controller includes a power supply module and an electronic controller, and configured to supply and apply DC power and corresponding voltage difference to the first pair of electrodes 151a and 151b and the second pair of electrodes 152a and 152b; the DC power can be, for ex ample but not limited to: 0.1V/cm, 0.33V/cm or 1V/cm. By applying the voltage difference to the first pair of electrodes 151a and 151b and the second pair of electrodes 152a and 152b, the first pair of electrodes 151a and 151b generate an electric field substantively parallel to the deformation direction on the elastic conductive layer 130, and the second pair of electrodes 152a and 152b generate an electric field substantively perpendicular to deformation direction on the elastic conductive layer 130, so as to provide the electrical stimulation on the cultured biological tissue, or to simulate the transmission of electrical signals between the cells in vivo.

Preferably, the fluidic channel layer 120 and the culture layer 140 includes biocompatible material, such as but not limited to, PDMS and is made by implementing molding or casting manner Preferably, the culture layer 140 includes biocompatible material, for example, but not limited to: PDMS, by implementing molding or casting manner. The above-mentioned fluidic channel layer 120, the elastic conductive layer 130, the culture layer 140, the first pair of electrodes 151a and 151b, and the second pair of electrodes 152a and 152b together form a core biocompatible reaction module 200 configured in the bioreactor 100.

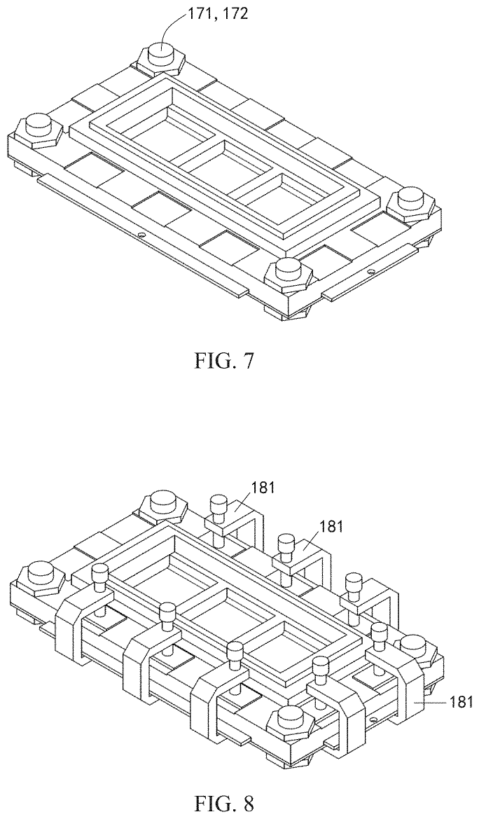

In FIG. 6, it demonstrates an upper case 160 assembled on the biocompatible reaction module 200. As shown in FIG. 6, the upper case 160 includes a plurality of manufacturing holes 161 cut therethrough and corresponding in position the assembling holes 112 of the lower base 110, a plurality of recessions 162 formed on peripheral edges thereof, and a protective cover positioning frame 163 placed in a central part thereof. The whole structure of the upper case 160 can cover a part or all of the frame structures 141 and position the culture layer 140. As shown in FIG. 7, multiple sets of bolts 171 and nuts 172 can be used to secure the lower base 110 and the upper case by locking the bolts 171 into the assembling holes 112 of the lower base 110 and the manufacturing holes 161 of the upper case 160, respectively. After the lower base 110 and the upper case 160 are secured, the fluidic channel layer 120, the elastic conductive layer 130 and the culture layer 140 included in the biocompatible reaction module 200 can be fastened.

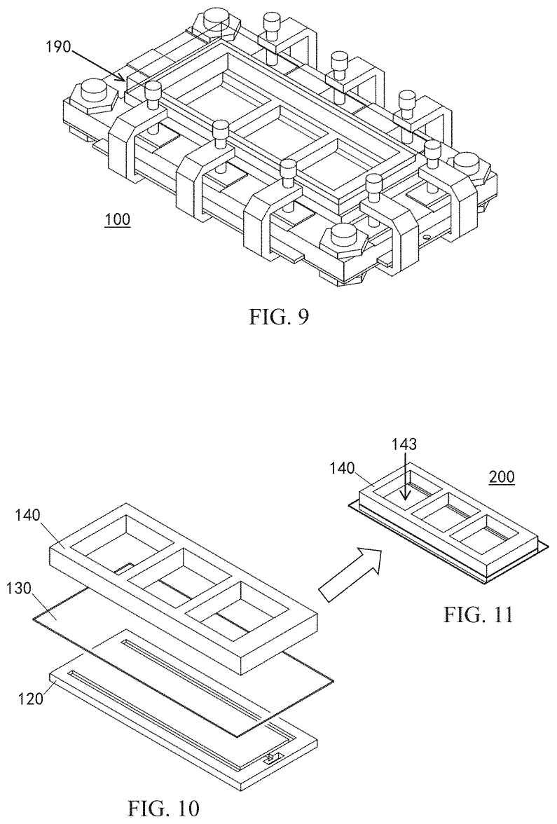

As shown in FIG. 8, multiple clamps 181 are respectively clamped in the recessions 162 of the upper case 160, to provide more clamping forces for the elastic conductive layer 130, the first pair of electrodes 151a and 151b and the second pair of electrodes 152a and 152b connected to the edges of the elastic conductive layer 130, so as to prevent the stretch amount of the elastic conductive layer 130 from causing slippage or displacement of the first pair of electrodes 151a and 151b and the second pair of electrodes 152a and 152b to result in poor electrical contact, thereby further enhancing the stretching efficiency of the elastic conductive layer 130 to generate sufficient stretch amount. Preferably, each clamp can be implemented by a C-shaped ring or C-shaped buckle to clamp in the corresponding recession 162, so as to secure the lower base 110 and the upper case 160. As shown in FIG. 9, a protective cover 190, which is light-transmittable or opaque, is placed on the positioning frame 163, to protect the biological tissues being cultured in vitro in the culture wells 143 from being contaminated by environmental impurities. As a result, the bioreactor 100 in accordance with the present invention can be completed according to above-mentioned operations.

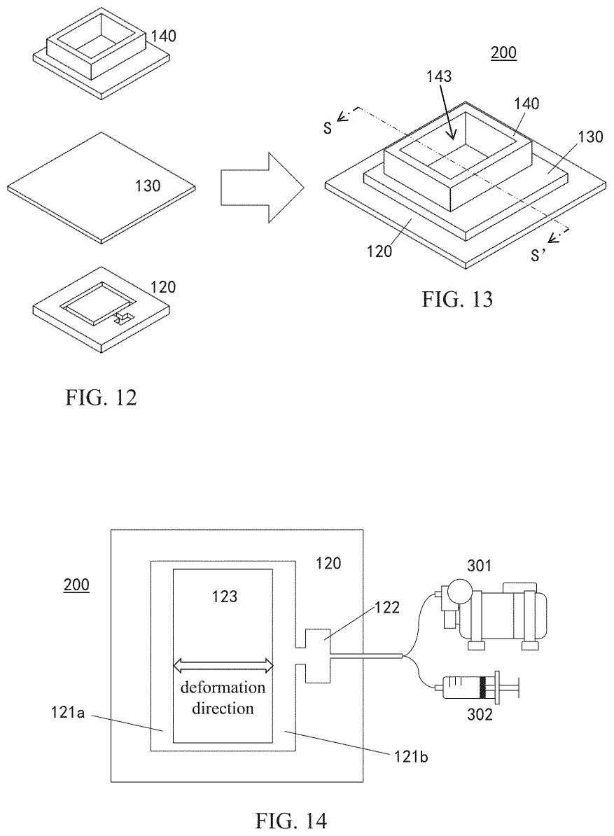

FIG. 10 is an exploded diagram illustrating most structural parts of a biocompatible reaction module, according to a second embodiment in accordance with the present invention; FIG. 11 is a schematic diagram illustrating a structural assembling operation for the biocompatible reaction module, according to the second embodiment in accordance with the present invention; FIG. 12 is an exploded diagram illustrating most structural parts of a biocompatible reaction module, according to a third embodiment in accordance with the present invention; and FIG. 13 is a schematic diagram illustrating a structural assembling operation of the biocompatible reaction module, according to the third embodiment in accordance with the present invention. The culture layer 140 of the biocompatible reaction module 200 can be designed to form an unlimited number of culture wells 143, for example, the biocompatible reaction module 200 can have three culture wells 143 as shown in FIGS. 10 and 11, or the biocompatible reaction module 200 can have single culture well 143 as shown in FIGS. 12 and 13.

The biocompatible reaction module 200 in accordance with the present invention includes the fluidic channel layer 120, the elastic conductive layer 130 and the culture layer 140 which are made by PDMS as main material. The fluidic channel layer 120 is bonded with the elastic conductive layer 130 by plasma bonding, and preferably, the elastic conductive layer 130 is adhered to the culture layer 140 by using biocompatible material such as PDMS as adhesive. The above-mentioned PDMS material, PDMS adhesive and oxygen plasma material are all highly biocompatible and are not toxic for the cell biological tissue cultured in vitro in the culture well 143, so the cultured biological tissue is not damaged even being in contact with these materials.

During the process of manufacturing the fluidic channel layer and the culture layer, the computer-assisted design and manufacturing (CAD/CAM) systems, such as but not limited to SolidWorks software, are used to design the design drawings of the forming molds for the fluidic channel layer and the culture layer, and then the design drawings are inputted to the computer numerical control (CNC) processing machine, which mills acrylic (PMMA) block material to form coarse mold blanks of the fluidic channel layer and the culture layer, and then the coarse mold blanks are polished to complete the forming molds. In next step, the agents A and B of liquid PDMS are blended in a specific ratio and stirred for a certain period of time, the mixture is poured into the forming mold. After being still placed for a period of time, the forming mold is defoamed, and then the forming mold and liquid PDMS are placed in a heater together to bake at a specific temperature for a period of time, so as to perform thermal curing of PDMS. After the baking process is completed, the PDMS can be cooled and demolded from the forming mold. As a result, the production of the fluidic channel layer and the culture layer is completed.

Preferably, the elastic bio-substrate included in the elastic conductive layer is made according to the following steps: (1) providing and placing a piece of PMMA substrate with a smooth and flat surface onto a spin coater machine; (2) blending the agents A and B of liquid PDMS in a specific ratio and stirring the mixture for a certain period of time, and then pouring the mixture on the PMMA substrate, and activating the spin coater machine to spin to cause the mixture flattened; (3) placing the PMMA substrate and the PDMS material coated on PMMA into a heater together to bake for a period of time at a specific temperature; (4), taking the PMMA substrate out of the heater, and taking the PDMS membrane from the PMMA.

A surface modification process is subsequently performed on the prepared PDMS membrane, by implementing the following steps: (1) immersing the PDMS membrane with, for example but not limited thereto, a sodium hydroxide (NaOH) aqueous solution of a specific molar concentration, and still placing the PDMS membrane at room temperature for about 5 to 7 hours, and preferably 6 hours; (2) washing the PDMS membrane with DI water to remove the remaining NaOH aqueous solution. Through above-mentioned steps, the surface layer of the PDMS membrane has wrinkled structures, corrugated structures or micro pore formed by etching effect resulted from the surface modification, to improve surface roughness, and facilitate the stretching operation of the PDMS membrane to generate more stretch amount. Furthermore, the wrinkled structures, corrugated structures are distributed substantially uniformly, so that light passing through by the PDMS membrane can be reflected and refracted substantively regularly, to facilitate subsequent imaging operation and make the PDMS membrane have excellent optical properties.

Next, an organic conductive membrane is deposited on the unilateral surface of the modified PDMS membrane by implementing the following steps: (1) attaching the modified PDMS membrane flat on, for example but not limited to, the bottom of a petri dish; (2) preparing ammonium persulfate (APS) aqueous solution and pyrrole (Py) aqueous solution with specific molar concentrations, respectively, and then placing these two solutions in a cooler to cool to a specific temperature; (3) blending the cooled APS aqueous solution and Py aqueous solution in equal volumes, and pouring the mixture the petri dish, and keeping the petri dish a certain temperature for a period of time for oxidative polymerization process in which the PPy organic conductive membrane is formed on the PDMS membrane to generate the PPy/PDMS membrane; (4) removing the APS aqueous solution and the Py aqueous solution from the petri dish, and washing the PPy/PDMS membrane with DI water; (5) using an ultrasonic oscillator clean the PPy powder remained on the surface of the PPy/PDMS membrane, so as to complete the production of the elastic conductive layer. The completed elastic conductive layer looks roughly black, and the PPy/PDMS membrane or the elastic conductive layer has a light transmittance in a range of from 30% up to 80% because the PDMS membrane has been processed by chemical surface modification in advance.

The back surface, on which no PPy organic conductive membrane is deposited, of the PPy/PDMS membrane is bonded on the front surface of the fluidic channel layer, and then oxygen plasma activation is performed on the back surface of the PPy/PDMS membrane and the front surface of the fluidic channel layer by placing the PPy/PDMS membrane and the fluidic channel layer into, for example but not limited to, the common cavity oxygen plasma machine, to bond the fluidic channel layer and the PPy/PDMS membrane tightly, so that the microchannel and the chamber can withstand considerable positive pressure or negative pressure during practical operation. Then, the surface of the PPy/PDMS membrane and the bottom of the culture layer are coated with PDMS adhesive and then placed into a heater to bake at a specific temperature for a period of time, so as to thermally cure the PDMS adhesive to improve the bonding strength of the plasma bonding interface.

Preferably, during the above pyrrole polymerization process, the thermosensitive polymer, for example but not limited to, monomers of NIPAm can be added to copolymerize with pyrrole, so as to finally obtain P(PPy-co-NIPAm) thermosensitive conductive membrane, which is polymerized on the PDMS membrane. The P(PPy-co-NIPAm) thermosensitive conductive membrane is biocompatible and has stretchability, conductivity and thermosensitivity, and the molecular chain of the thermosensitive conductive membrane can alter hydrophilicity of the surface layer in response to the change of a temperature. For example, when the temperature increases, the molecular chain of the thermosensitive conductive membrane tends to form a hydrophobic coiling flat conformation, to which is easier for cell to attach. When the temperature decreases, the molecular chain of the thermosensitive conductive membrane tends to form a hydrophilic stretched straight-chain conformation, the extracellular matrix is pushed away and the cells can be removed from the surface without damaging structures of the cells, and the above process is called the cooling and detaching process. Through the cooling and detaching process, the tissues cultured and attached on the thermosensitive conductive layer can be removed from thermosensitive conductive layer without damage.

Preferably, the P(PPy-co-NIPAm) thermosensitive conductive membrane can be deposited on the surface layer of the PDMS membrane (as the substrate) by implementing the following steps: (1) attaching the modified PDMS membrane flat on, for example but not limited to, the bottom of the petri dish; (2) preparing the APS aqueous solution, PPy aqueous solution and NIPAm aqueous solution with specific molar concentrations; (3) blending the PPy aqueous solution and the NIPAm aqueous solution and adding a specific amount of Temed solution into the mixture, and pouring the mixture into the petri dish and using a vortex mixer to shake the mixture for well mix; (4) adding the APS aqueous solution into the mixture and blending the mixture well, and placing the mixture at room temperature for a period of time, so as to form the P(PPy-co-NIPAm) thermosensitive conductive membrane on the PDMS membrane; (5) removing the aqueous solution from the petri dish, and washing the P(PPy-co-NIPAm)/PDMS membrane with DI water; (6) placing the P(PPy-co-NIPAm)/PDMS membrane into a heater for heating at a specific temperature for a period of time, so as to complete the production of the elastic conductive layer.

FIG. 14 is a schematic diagram illustrating the connection and communication between the bioreactor in accordance with the present invention and an external fluid driving equipment. The bioreactor 100 in accordance with the present invention includes microchannels 121a and 121b and a chamber 122 in the fluidic channel layer 120, and preferably, the microchannels 121a and 121b and the chamber 122 are filled with suitable fluid media such as gas or liquid, and the chamber 122 is connected with the external fluid driving equipment, such as but not limited to, an air pump 301 or a syringe 302. The external fluid driving equipment is used to pump or inject the fluid media filled in the fluidic channel layer 120, to enable the elastic conductive layer 130 above the fluidic channel layer 120 to produce corresponding stretch or contraction mechanical action, and the deformation direction is perpendicular to the microchannels 121a or 121b.

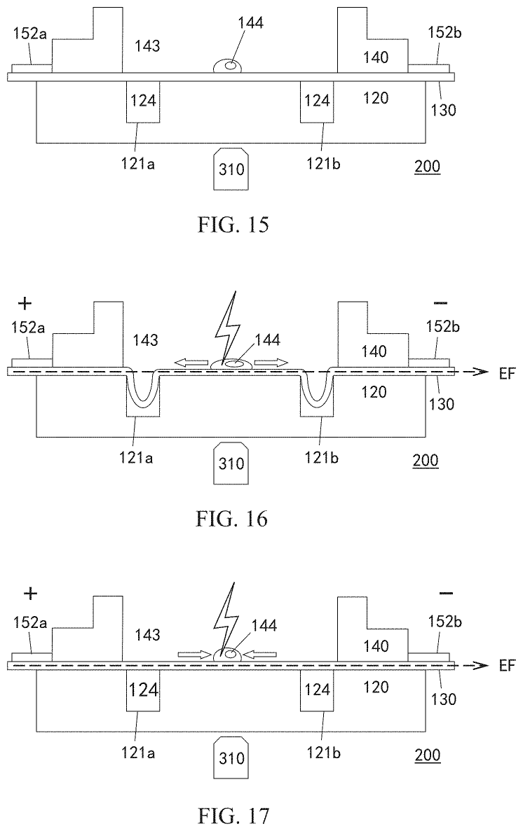

FIGS. 15 to 17 are a series of schematic cross-sectional diagrams acquired along a section line SS' shown in FIG. 13 showing an operating process of a biocompatible reaction module of a bioreactor in accordance with the present invention. As shown in FIG. 15, when the biocompatible reaction module 200 in accordance with the present invention is not activated, the elastic conductive layer 130 is in a neutral status. Each of the fluidic channel layer 120 and the elastic conductive layer 130 can be made by light-transmittable materials, so that a user can observe the structure and cell type of the biological tissue 144 cultured in vitro in the culture well 143 of the culture layer 140 and on the elastic conductive layer 130 through the imaging equipment 310 installed below the biocompatible reaction module 200. For example, the imaging equipment 310 is preferably a microscope, an inverted microscope, and a scanning electron microscope (SEM) and so on.

As shown in FIG. 16, when the fluid driving equipment commences to remove the fluid media 124 out of the fluidic channel layer 120, the elastic conductive layer 130 is sucked down into the microchannels 121a and 121b under the principle of continuity during the process of pumping the fluid media 124 out of the microchannels 121a and 121b, so that the elastic conductive layer 130 on the experiment and observation platform 123 is stretched toward the vertical walls 142 to have a deformation, and the biological tissue 144 cultured and attached on the elastic conductive layer 130 is also stretched together. Through the imaging equipment 310 located below the biocompatible reaction module 200, the user can observe the structural change and reaction of the biological tissue 144 being mechanically stimulated, such as being stretched. As shown in FIG. 17, when the fluid driving equipment injects the fluid media 124 into the fluidic channel layer 120, the fluid media 124 flows into the microchannels 121a and 121b again, so that the elastic conductive layer 130, which is previously stretched to tighten, loses the tension and begins to contract, and the biological tissue 144 attached on the elastic conductive layer 130 synchronously returns to the initial state from the mechanically-stimulated state.

Preferably, a width and a depth of each of the microchannels 121a and 121b is configured to be adjustable, so that the stretch amount for the elastic conductive layer 130 and the elastic bio-substrate can be adjusted through an adjustment to the width or the depth; for example, when the depth or groove depth of the microchannels 121a and 121b is adjusted to 0.5 mm, 1 mm or 1.5 mm, it respectively causes 6.5%, 9% or 13% of stretch amount for the elastic conductive layer 130, and the biological tissue 144 cultured and attached on the elastic conductive layer 130 is driven to stretch simultaneously. The stretching process can be achieved by, for example, using the second pair of electrodes 152a and 152b to apply a surface field EF to the biological tissue 144 on the elastic conductive layer 130, and the surface field EF is substantively parallel to the deformation direction, so as to apply the combined stimulations including the mechanical stimulation and the electrical stimulation to the biological tissue 144.

The biocompatible reaction module 200 in accordance with the present invention is driven by fluid, and preferably, the fluid media 124 filled in the microchannels 121a and 121b is gas or fluid. Because the pressure wave inside the fluid conducts at a high speed, the fluid media 124 can quickly respond to the positive pressure or the negative pressure generated by the fluid driving equipment, the elastic conductive layer 130 can be stretched to the limit thereof in a very short time, thereby generating a pulse-like rapid stretch. The electronic controller or the electronic control system can be configured to generates a cyclic rapid stretch, and can also control the stretching speed, so as to faithfully simulate the actual action of the biological tissue in the organism.

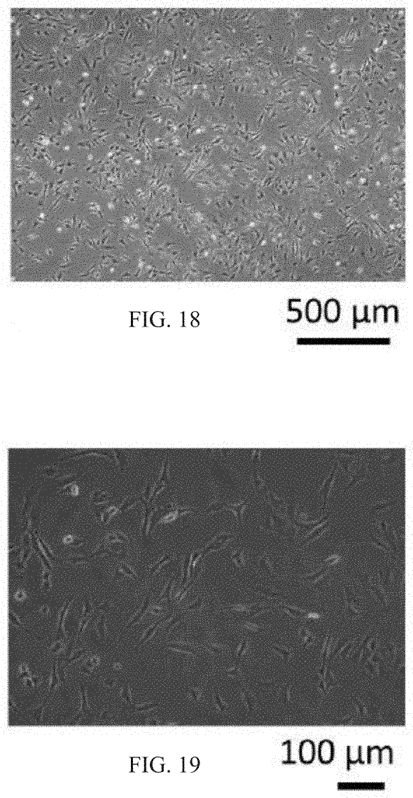

FIG. 18 is a microscopic image observed by using an inverted microscope with 4× objective lens and showing the biological tissue cell cultured on the elastic conductive layer of the bioreactor in accordance with the present invention; and FIG. 19 is a microscopic image observed by using an inverted microscope with 10× objective lens and showing the biological tissue cell cultured on the elastic conductive layer of the bioreactor in accordance with the present invention. As described above, the elastic conductive layer 130 (such as the PPy/PDMS membrane or P(PPy-co-NIPAm)/PDMS membrane) in accordance with the present invention is processed by chemical surface modification, so that light passing through the elastic conductive layer 130 can be regularly reflected and refracted, and when the imaging equipment 310, such as the inverted microscope with 4× objective lens or 10× objective lens, is used to observe, the biological tissue 144 cultured on the elastic conductive layer 130 renders a clear and identifiable image on the imaging equipment 310, as shown in FIGS. 18 and 19.

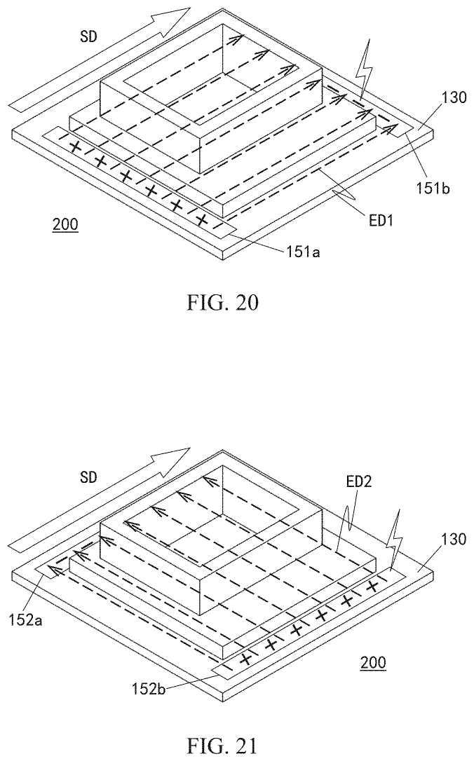

FIG. 20 is a schematic diagram illustrating a biocompatible reaction module generating the combined stimulation in an electrical field direction substantively parallel to the deformation direction; and FIG. 21 is a schematic diagram illustrating the biocompatible reaction module generating the combined stimulation in an electrical field direction perpendicular to the deformation direction, according to the present invention. In the biocompatible reaction module 200 in accordance with the present invention, the elastic conductive layer 130 is to be stretched or contracted along the deformation direction, and in the first embodiment, the deformation direction means, for example but not limited to, the direction substantively perpendicular to the direction of the microchannel 121a or 121b; when the voltage difference is applied to the first pair of electrodes 151a and 151b and the second pair of electrodes 152a and 152b connected to the periphery of the elastic conductive layer 130, the first pair of electrodes 151a and 151b and the second pair of electrodes 152a and 152b generate the electric fields in different directions on the elastic conductive layer 130, respectively.

As shown in FIG. 20, the first pair of electrodes 151a and 151b generates the first electric field on the elastic conductive layer 130 along the first electrical field direction ED1 after being applied by voltage difference, and the first electrical field direction ED1 is substantively parallel to the deformation direction SD. As shown in FIG. 21, the second pair of electrodes 152a and 152b generates the second electric field on the elastic conductive layer 130 along the second electrical field direction ED2 after being applied by the voltage difference, and the second electrical field direction ED2 in substantively perpendicular to the deformation direction SD. Through the imaging equipment 310 installed below the biocompatible reaction module 200, a user can observe various structural changes and reactions of the biological tissue 144 after the combined stimulations including one of the mechanical stimulation, the electrical stimulation and a combination thereof, is performed on the biological tissue.

By using the bioreactor in accordance with the present invention, the operator can freely control at least one of stretching speed, stretching frequency, stretching period, stretch amount, stretching mode, pulse stretching, cyclic stretching, forced stretching, natural contraction, voltage level, current level, staggered stimulation, cyclic stimulation, combined stimulations and stimulation mode, so that the operator can freely plan, combine and apply these control factors to observe the change and response of the biological tissue stimulated in the actual experiment with different control factors.

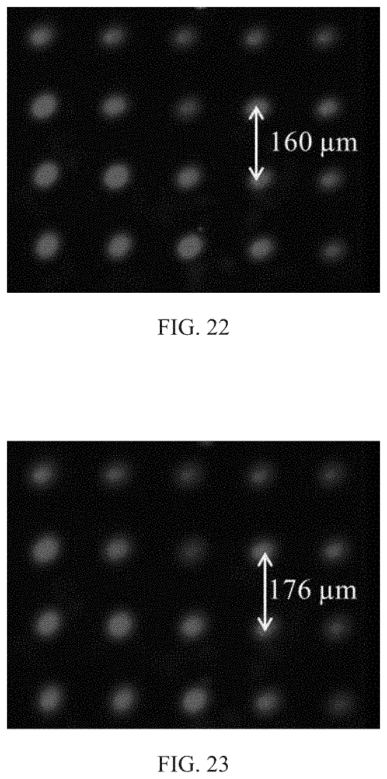

FIG. 22 is a microscope image showing arrangement of array of fluorescent markers before the elastic conductive layer printed with the array of fluorescent markers is stretched; and FIG. 23 is a microscope image showing the change in arrangement of the array of fluorescent markers after the elastic conductive layer printed with the array of fluorescent markers is stretched. According to the present invention. In order to measure the stretch amount of the elastic conductive layer 130, the present invention further applies the micro contact printing technology to print the array of fluorescent markers on the elastic conductive layer 130, and analyze the change in positions or interval of the fluorescent markers before and after the stretching operation, so as to effectively calculate the stretch amount of the elastic conductive layer 130. Preferably, the stretch amount is controlled or set at 10%, because the stretch amount limit for the muscle biological tissue is approximately 10%, and the stretch amount exceeding 10% may cause damage to the muscle biological tissue.

The micro contact printing is an application of soft lithography technology, and the micro contact printing process mainly includes three stages of template preparation, inking operation, and ink transfer. First of all, lithography is used to produce a template including a pattern of micro-seal, and then the template is used to make a micro-seal. The micro seal has wrinkled structures formed thereon and carrying the pattern to be printed. After micro-seal is stained with fluorescent ink, the micro-seal directly contacts the elastic conductive layer 130, to transfer the patterns of the array of fluorescent markers to be printed to the elastic conductive layer 130, just like nanotransfer printing. The present invention uses BSA standard mixed with rhodamine B as the fluorescent dye. As shown in FIG. 22, the interval between the fluorescent markers of the elastic conductive layer is measured as 160 μm before stretching; as shown in FIG. 23, the interval between the fluorescent markers is changed up to 176 μm after stretching, so the stretch amount of the elastic conductive layer is calculated as 10%.

According to electrical reliability test result, the elastic conductive layer 130 is measured to has a stable electrical resistance lower than 20 kΩ when being sketched within a certain stretch amount, such as in a range of 30% up to 40%. Therefore, as long as the stretch amount does not exceed 40%, the elastic conductive layer 130 is substantively maintained at the status with electrical resistance of less than 20 kg. In particular, the elastic conductive layer 130 (such as PPy/PDMS membrane or P(PPy-co-NIPAm)/PDMS membrane) processed by the chemical surface modification for about 6 hours expresses the most stable electrical resistance and biological suitability, and the micro pores formed by the chemical surface modification can improve the optical properties of the elastic conductive layer 130, so as to facilitate the use of the imaging equipment to observe the cultured biological tissues or cells. In a practical experiment, the mouse muscle fibroblast cell (C2C12) is actually cultured on the elastic conductive layer 130, and the test result of the MTT assay performed on the cultured cells proves that elastic conductive layer 130 improves the biological activity of the surface cells.

The bioreactor proposed in the present invention is designed based on the biocompatible material and uses the fluid-driven method to drag the elastic conductive layer, in which the biological tissue is cultured, into the microchannel by a negative pressure, and to stretch the elastic conductive layer to provide the stretching stimulation to the biological tissue, and perform the electrical stimulation to the biological tissue by configuring multiple pairs of electrodes on the edge of the elastic conductive layer, so as to provide the combined stimulations to the biological tissue. The bioreactor in accordance with the present invention is simple in design and beneficial in space and cost, and has the culture zone providing a large culture space, and more importantly, the culture zone is stretched very uniformly to provide uniform stimulation to the biological tissue; furthermore, the bioreactor in accordance with the present invention is able to combine the stretching stimulation and the electrical stimulation. In general, the bioreactor in accordance with the present invention is good in design with advantages of a large culture space, low cost, convenient production, easy to carry, and less space.

The present invention proposes a bioreactor which has a simple design and reasonable cost and effectively provides the various stimulations including the electrical stimulation, the stretching stimulation or a combination thereof, to the cells or the biological tissue, the operator can plan and try different combinations of stimulations to conduct more, broader, deeper, more complex, and more detailed researches on human biological tissue. The bioreactor in accordance with the present invention can be applied to, for example but not limited to: biological tissue engineering, regeneration medicine, cell therapy, biomedical technology, clinical application, cell biology, life medicine, materials engineering, bionic technology, pathological model construction and other fields, and is able to make substantial contributions to the research and development of various fields.

There are further embodiments provided as follows.

Embodiment 1: A bioreactor includes a fluidic channel layer including a set of channels configured to generate a suction caused by a negative pressure or a retrieval force caused by a positive pressure; an elastic conductive layer configured with a pair of electrodes, configured on the fluidic channel layer, driven by the suction or the retrieval force to have a deformation toward a deformation direction, and receiving a voltage difference by the pair of the electrodes to form an electrical field along an electrical field direction; and a culture layer configured on the elastic conductive layer and providing for a biological tissue to culture in vitro on the elastic conductive layer.

Embodiment 2: The bioreactor as described in Embodiment 1, further includes one of components as follows: the fluidic channel layer including a chamber connected with the set of channel; the set of channel including at least one channel filled with a fluid media; the elastic conductive layer further including an elastic bio-substrate having a surface layer and an organic conductive membrane deposited on the surface layer, wherein the elastic bio-substrate is capable of producing a stretch amount up to 40%; the set of channel included in the fluidic channel layer having a width or a depth that is configured to be adjustable, so as to control the stretch amount for the elastic bio-substrate through an adjustment to the width or the depth; the culture layer including a culture zone having a plurality of vertical walls that form at least one culture well together with the elastic conductive layer beneath the culture layer; a lower base including a plurality of assembling holes and a central slot providing for the fluidic channel layer to embed therein; an upper case configured on the culture layer and including a central opening, a plurality of recessions for clamps formed and distributed close to edges, a positioning frame formed around the central opening, and a plurality of manufacturing holes aligned to the plurality of assembling holes respectively in position; a protective cover placed on the positioning frame to cover the at least one culture well; a plurality of first fasteners configured to assemble the lower base, the fluidic channel layer, the elastic conductive layer, the culture layer and the upper case in order together by fastening through the plurality of assembling holes and the plurality of assembling holes that are aligned with each other; a plurality of second fasteners including a plurality of bolts and a plurality of nuts configured to assemble the lower base, the fluidic channel layer, the elastic conductive layer, the culture layer and the upper case in order together by inserting the plurality of bolts through the plurality of assembling holes and the plurality of assembling holes that are aligned with each other and screwing the plurality of bolts into the plurality of nuts on opposite side; a plurality of hinge connectors configured at respective edges of the lower base and the upper case to render the upper case to pivotally revolve with respect to the lower base, so as to clip the fluidic channel layer, the elastic conductive layer, and the culture layer in order together by fastening the lower base and the upper case; a plurality of clamps clamping in the plurality of recessions to secure the lower base and the upper case together and to provide more clamping force for the elastic conductive layer and the pair of electrodes; a fluid driving equipment having an output port connected with the chamber to generate the suction to cause the deformation by pumping the fluid media out of the set of channel through the chamber or to generate the retrieval force to cause the deformation by injecting the fluid media into the set of channel through the chamber; and an electronic controller including a power supplier module and an electronic controller module to supply the voltage difference to the pair of electrodes to form the electrical field.

Embodiment 3: The bioreactor as described in Embodiment 2, the elastic bio-substrate has the surface layer that is processed by a surface modification which is a surface modification processing selected from one of an alkaline aqueous solution immersion processing, an acidic aqueous solution immersion processing, an oxidant solution immersion processing, a plasma processing, an ion beam processing, a high energy electromagnetic waves processing, and a combination thereof, and therefore the surface layer has multiple wrinkled structures, corrugated structures and micro pores that are formed by etching effect resulted from the surface modification to improve surface roughness and to produce activated functional groups.

Embodiment 4: The bioreactor as described in Embodiment 2, the organic conductive membrane includes material selected from one of a polypyrrole (PPy), a polythiophene (PT), a polyaniline (PANi), a polyphenylene sulfide (PPS), and a combination thereof, and the fluidic channel layer, the elastic bio-substrate and the culture layer includes material selected from one of a polydimethylsiloxane (PDMS), a polyurethane (PU), a rubber, an elastomer, and a combination thereof, and the organic conductive membrane is deposited onto the elastic bio-substrate by implementing an oxidant polymerization process.

Embodiment 5: The bioreactor as described in Embodiment 2, the organic conductive membrane has a surface is printed with an array of florescent markers consisting of a plurality of florescent markers by performing a micro contact printing so as to measure the stretch amount caused by the deformation, and has an electrical resistance less than 20 kiloohms within the stretch amount of less than 40%.

Embodiment 6: The bioreactor as described in Embodiment 2, the fluidic channel layer and the elastic conductive layer are light-transmittable, and the elastic conductive layer has a light transmittance in a range of from 30% up to 80%, which renders an identifiable image for the biological tissue to be formed on an imaging equipment.

Embodiment 7: The bioreactor as described in Embodiment 1, the elastic conductive layer is bonded to the fluidic channel layer by implementing a plasma bonding, the elastic conductive layer is adhered to the culture layer by using a biocompatible adhesive.

Embodiment 8: The bioreactor as described in Embodiment 1, the deformation direction is substantively parallel to the electrical field direction or the deformation direction is substantively perpendicular to the electrical field direction.

Embodiment 9: A bioreactor includes a fluidic channel layer including a set of channels configured to generate a suction caused by a negative pressure or a retrieval force caused by a positive pressure; an elastic conductive layer configured with a pair of electrodes, configured on the fluidic channel layer, driven by the suction or the retrieval force to have a deformation toward a deformation direction, receiving a voltage difference by the pair of the electrodes to form an electrical field along an electrical field direction, and altering a degree of surface hydrophilicity in response to a change of a temperature; and a culture layer configured on the elastic conductive layer and providing for a biological tissue to culture in vitro on the elastic conductive layer.

Embodiment 10: The bioreactor as described in Embodiment 9, the elastic conductive layer further includes an elastic bio-substrate and a thermosensitive conductive membrane deposited on the elastic bio-substrate.

Embodiment 11: The bioreactor as described in Embodiment 10, the thermosensitive conductive membrane further includes material selected from one of a polypyrrole (PPy), a polythiophene (PT), a polyaniline (PANi), a polyphenylene sulfide (PPS), a poly(N-isopropylacrylamide) (NIPAm), and a combination thereof.

While the disclosure has been described in terms of what are presently considered to be the most practical and preferred embodiments, it is to be understood that the disclosure need not be limited to the disclosed embodiments. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present disclosure which is defined by the appended claims.

What is claimed is:

1. A bioreactor comprising:
a fluidic channel layer comprising a set of channels configured to generate a suction caused by a negative pressure or a retrieval force caused by a positive pressure;
an elastic conductive layer configured with a pair of electrodes, configured on the fluidic channel layer, driven by the suction or the retrieval force to have a deformation toward a deformation direction, and receiving a voltage difference by the pair of the electrodes to form an electrical field along an electrical field direction; and
a culture layer configured on the elastic conductive layer and providing for a biological tissue to culture in vitro on the elastic conductive layer.

2. The bioreactor as claimed in claim 1, further comprising one of components as follows:
the fluidic channel layer comprising a chamber connected with the set of channel;
the set of channel comprising at least one channel filled with a fluid media;
the elastic conductive layer further comprising an elastic bio-substrate having a surface layer and an organic conductive membrane deposited on the surface layer, wherein the elastic bio-substrate is capable of producing a stretch amount up to 40%;
the set of channel comprised in the fluidic channel layer having a width or a depth that is configured to be adjustable, so as to control the stretch amount for the elastic bio-substrate through an adjustment to the width or the depth;
the culture layer comprising a culture zone having a plurality of vertical walls that form at least one culture well together with the elastic conductive layer beneath the culture layer;
a lower base comprising a plurality of assembling holes and a central slot providing for the fluidic channel layer to embed therein;
an upper case configured on the culture layer and comprising a central opening, a plurality of recessions for clamps formed and distributed close to edges, a positioning frame formed around the central opening, and a plurality of manufacturing holes aligned to the plurality of assembling holes respectively in position;
a protective cover placed on the positioning frame to cover the at least one culture well;
a plurality of first fasteners configured to assemble the lower base, the fluidic channel layer, the elastic conductive layer, the culture layer and the upper case in order together by fastening through the plurality of assembling holes and the plurality of assembling holes that are aligned with each other;
a plurality of second fasteners including a plurality of bolts and a plurality of nuts configured to assemble the lower base, the fluidic channel layer, the elastic conductive layer, the culture layer and the upper case in order together by inserting the plurality of bolts through the plurality of assembling holes and the plurality of assembling holes that are aligned with each other and screwing the plurality of bolts into the plurality of nuts on opposite side;
a plurality of hinge connectors configured at respective edges of the lower base and the upper case to render the upper case to pivotally revolve with respect to the lower base, so as to clip the fluidic channel layer, the elastic conductive layer, and the culture layer in order together by fastening the lower base and the upper case;
a plurality of clamps clamping in the plurality of recessions to secure the lower base and the upper case together and to provide more clamping force for the elastic conductive layer and the pair of electrodes;
a fluid driving equipment having an output port connected with the chamber to generate the suction to cause the deformation by pumping the fluid media out of the set of channel through the chamber or to generate the retrieval force to cause the deformation by injecting the fluid media into the set of channel through the chamber; and
an electronic controller comprising a power supplier module and an electronic controller module to supply the voltage difference to the pair of electrodes to form the electrical field.

3. The bioreactor as claimed in claim 2, wherein the elastic bio-substrate has the surface layer that is processed by a surface modification which is a surface modification processing selected from one of an alkaline aqueous solution immersion processing, an acidic aqueous solution immersion processing, an oxidant solution immersion processing, a plasma processing, an ion beam processing, a high energy electromagnetic waves processing, and a combination thereof, and therefore the surface layer has multiple wrinkled structures, corrugated structures and micro pores that are formed by etching effect resulted from the surface modification to improve surface roughness and to produce activated functional groups.

4. The bioreactor as claimed in claim 2, wherein the organic conductive membrane comprises material selected from one of a polypyrrole (PPy), a polythiophene (PT), a polyaniline (PANi), a polyphenylene sulfide (PPS), and a combination thereof, and the fluidic channel layer, the elastic bio-substrate and the culture layer comprises material selected from one of a polydimethylsiloxane (PDMS), a polyurethane (PU), a rubber, an elastomer, and a combination thereof, and the organic conductive membrane is deposited onto the elastic bio-substrate by implementing an oxidant polymerization process.

5. The bioreactor as claimed in claim 2, wherein the organic conductive membrane has a surface is printed with an array of florescent markers consisting of a plurality of florescent markers by performing a micro contact printing so as to measure the stretch amount caused by the deformation, and has an electrical resistance less than 20 kiloohms within the stretch amount of less than 40%.

6. The bioreactor as claimed in claim 2, wherein the fluidic channel layer and the elastic conductive layer are light-transmittable, and the elastic conductive layer has a light transmittance in a range of from 30% up to 80%, which renders an identifiable image for the biological tissue to be formed on an imaging equipment.

7. The bioreactor as claimed in claim 1, wherein the elastic conductive layer is bonded to the fluidic channel layer by implementing a plasma bonding, the elastic conductive layer is adhered to the culture layer by using a biocompatible adhesive.

8. The bioreactor as claimed in claim 1, wherein the deformation direction is substantively parallel to the electrical field direction or the deformation direction is substantively perpendicular to the electrical field direction.

9. A bioreactor comprising:
a fluidic channel layer comprising a set of channels configured to generate a suction caused by a negative pressure or a retrieval force caused by a positive pressure;
an elastic conductive layer configured with a pair of electrodes, configured on the fluidic channel layer, driven by the suction or the retrieval force to have a deformation toward a deformation direction, receiving a voltage difference by the pair of the electrodes to form an electrical field along an electrical field direction, and altering a degree of surface hydrophilicity in response to a change of a temperature; and
a culture layer configured on the elastic conductive layer and providing for a biological tissue to culture in vitro on the elastic conductive layer.

10. The bioreactor as claimed in claim 9, wherein the elastic conductive layer further comprises an elastic bio-substrate and a thermosensitive conductive membrane deposited on the elastic bio-substrate.

11. The bioreactor as claimed in claim 10, wherein the thermosensitive conductive membrane further comprises material selected from one of a polypyrrole (PPy), a polythiophene (PT), a polyaniline (PANi), a polyphenylene sulfide (PPS), a poly(N-isopropylacrylamide) (NIPAm), and a combination thereof.
